(19) United States
(12) Patent Application Publication
(10) Pub. No.: US 2012/0022508 A1
(43) Pub. Date: Jan. 26, 2012

(54) METHOD AND ORIENTATION DEVICE FOR MARKING A PREDETERMINED GUIDE PATH OF A MEDICAL INSTRUMENT

(76) Inventors: Patrick Gross, Langensendelbach (DE); Martin Requardt, Nuernberg (DE); Anke Weissenborn, Weil am Rhein (DE)

(21) Appl. No.: 13/178,863

(22) Filed: Jul. 8, 2011

(30) Foreign Application Priority Data
Jul. 22, 2010 (DE) 10 2010 031 943.0

(51) Int. Cl. A61B 17/00

(52) U.S. Cl. 606/1

(57) ABSTRACT

In a method for marking a predetermined guide path of a medical instrument in particular a needle, at least one guide path having an angle relative to vertical is indicated by means of at least one adjustable indicator unit such as a light source, and a one-dimensional or two-dimensional inclination sensor is used to adjust the at least one indicator unit with regard to the angle relative to vertical.

### FIELD OF THE INVENTION

The invention concerns a method for marking a predetermined guide path of a medical instrument, in particular of a needle, wherein by means of at least one adjustable indicator (in particular a light source), at least one guide path having an angle relative to vertical is made visible, as well as an associated orientation device.

### DESCRIPTION OF THE PRIOR ART

Minimally-invasive procedures that (for example) are implemented with a medical instrument (such as puncture needle or a biopsy needle) are frequently planned—with regard to the entrance point to be selected and the entrance direction, summarized as a guide path—using images acquired with a computed tomography device (CT device) or a magnetic resonance (MR) device. It has long been typical that a treating personnel had to conduct the actual procedure without further assistance according to his or her personal experience after the planning was concluded. For this purpose, the needle would possibly initially be inserted only a bit in order to then check (by means of additional image exposures) whether the trajectory coincides with the planned trajectory. Nevertheless, it frequently was necessary for the needle to be applied repeatedly.

In order to rectify this drawback, orientation devices were developed that were intended to assist the treating personnel in transferring the planned guidance path to the actual procedure. Such a device is known from DE 195 01 069 A1, for example. The device makes use of at least two sources of electromagnetic radiation that emit intersecting beams, with the intersection space of the beams marking the provided guidance path. Laser beams are used for this purpose. For example, a cylindrical lens can be used to generate a light fan situated in one plane.

In order to be able to actually use such an orientation device, it should ideally have a known alignment, in particular an alignment registered with the image acquisition device that is used. It is then possible, for example, to calculate how a given indicator unit must be oriented in order to be able to correctly specify the guide path based on the image exposures. It is often not possible (particularly in the use of magnetic resonance) to implement the procedure within the image acquisition device, so that it is necessary to move the patient out of the image acquisition device again (for example by means of a movable patient table) in order to ultimately implement the procedure.

In a known solution it is typical to use a laser marking that is present in the image acquisition device, for example to mark the central sagittal slice direction and the axial slice direction, for example as a crosshair. It is not difficult to move the patient table, to cause a known point in the image to be located at the axial plane as it is marked (indicated) by the laser. Treating personnel frequently use this procedure in order to determine the axial plane in which the planned entrance point lies, and to estimate the distance that it would have to travel in the axial direction along the patient skin. However, this procedure is not accurate since only one estimation is made as to where the entrance point should lie along the axial plane. However, the angles that determine the entrance direction are likewise selected only with visual judgment and with a free hand.

Fluoroscopy systems with a C-arm are known that have an integrated laser that can indicate the predetermined entrance point and additionally can indicate an entrance direction in the plane of the skin by orienting an integrated laser. However, the angle cannot be indicated relative to vertical with such systems.

Furthermore, grids or markers that are visible in magnetic resonance can be used in order to mark entrance points on the skin, but the angles indicated in the entrance direction can hereby in turn be determined only with visual perception.

Navigation systems were also known that use different cameras or radio-frequency markers in order to determine the correct position and angular position of the instrument; but these solutions are very expensive.

This is also the case for robot arms used within the image acquisition device. Such robot arms also introduce additional hardware and complexity into the operating procedure and present significant sterilization difficulties in addition to their high cost.

Orientation devices that are more advantageous to realize (such as those mentioned in the aforementioned DE 195 01 069 A1) have disadvantages, namely that it can easily occur that the stands supporting the indicator unit are not exactly aligned (in particular relative to vertical). Because the optical indicator unit are arranged at a significant distance from the actual procedure area, a slight angular deviation from vertical can already precipitate pronounced indication errors.

### SUMMARY OF THE INVENTION

An object of the present invention is to provide a method and orientation device to mark a predetermined guide path for an instrument in which error in the marking relative to vertical is largely avoided, or at least substantially reduced.

This object is achieved in a method of the aforementioned type wherein, according to the invention, a one-dimensional or two-dimensional inclination sensor is used to adjust the at least one indicator unit with regard to the angle relative to vertical.

According to the invention, an inclination sensor is used (that determines the angle relative to vertical, thus relative to the direction of the gravitational force) either to correct the indicated angle given an incorrect position/incorrect registration, or to directly determine the angle relative to vertical. Such an inclination sensor that can be associated with an indicator unit is then arranged in immediate proximity to the indicator unit, or at least in a fixed, known geometric relationship with the indicator unit. In this way, error-prone incorrect positions can no longer have an influence on the precision of the marking since vertical is known at any point in time and can be used as an actual reference.

While it is conceivable in principle to use a conventional spirit level (for example operating with a gas bubble in a liquid) as an inclination sensor, according to the invention an acceleration sensor (accelerometer) is preferably used because such a device delivers angle information with a high degree of accuracy and precision. Such an accelerometer can advantageously embody a MEMS (Micro Electromechanical System, also called a microsystem), thus a miniaturized assembly that (for example) can include multiple accelerometers. As mentioned, the angle can be adjusted relative to vertical using only the measurement data of the inclination sensor. This means that the determined vertical is used as an absolute reference that is consequently completely independent of the actual alignment of the indicator unit or of an element that supports (mounts) the indicator unit. In any case vertical in the coordinate system of the indicator unit is always known, such that an indicator unit that is also independent of other components of an orientation device (possibly freely movable indicator unit) can be realized that nevertheless can in principle correctly deliver an indication that serves as the marking of the correct angle relative to vertical.

For example, the correct realization can be such that the previously determined direction of the guide path is described by a rotation in the angle describing the horizontal plane and the angle relative to vertical. The adjustment of an orientation device can ultimately also take place in such a sequence. For example, initially a rotation in the horizontal is produced, whereupon a corresponding adaptation of an additional indicator unit which indicates the inclination relative to vertical, can take place.

In a further embodiment of the method according to the invention, the inclination sensor can be combined with an instrument guide (in particular a needle guide). This is an exemplary embodiment in which part of an orientation device can be achieved as a freely movable part without a fixed registration. In such an embodiment, with a laser system or the like, only the predetermined entrance point and one entrance direction into the plane of the skin (frequently essentially the horizontal) is indicated, for example by a corresponding projection onto the skin of the patient in the procedure area. The inclination sensor, which provides data or a signal indicating the direction of the vertical relative to the orientation of the instrument guide (and consequently relative to the instrument), is contained in the instrument guide (in particular in the needle guide). Depending on how the actual orientation of the instrument guide (and consequently of the instrument) relative to vertical are aligned in comparison to the desired orientation (thus the angle relative to vertical), a corresponding indicator unit (for example also at the instrument guide itself) can be activated. Not just one, but multiple, optical indicator units may be provided, for example an acoustic indicator unit in which the treating personnel can follow from the output tone how the instrument is to be moved in order to adopt the correct, predetermined entrance direction. As mentioned, a complete orientation aid is provided when an optical indicator unit indicates the predetermined entrance point of the instrument and/or a direction in the horizontal plane together with such an accurate item of information about the angle relative to vertical.

Alternatively or additionally, the inclination sensor can be arranged on (or in a fixed geometric relation to) an optical indicator unit (in particular an indicator unit that radiates a laser fan) is used. For example, in this context at least two light fans (in particular two laser fans) can be set independently. A laser fan can be aligned vertically and can be adjusted via a rotation in the horizontal plane so that it indicates the angle to be selected in the horizontal plane. The second fan can then be adjusted under consideration of the data of the inclination sensor so that the correct inclination relative to vertical is indicated. A clearly defined line that corresponds to the guide path then results in the overlap region of the two fans. Given such adjustments it need only be ensured that the entrance point (indicated by the cross projected onto the skin of the patient) also does shift in the displacement of the second fan. For example, compensation means can be provided for this purpose. In particular, a movement in an additional degree of freedom can be implemented to compensate for a displacement of the entrance point.

One possible workflow within the scope of the method according to the invention can be as follows, for example. Image data of the patient are initially acquired, for example by means of a CT device or an MR device. The planning of the procedure now takes place in these image data in that the guide path is determined which is ultimately defined by the entrance point and the entrance device. The coordinates of the guide path are then translated into the coordinate system of the orientation device being used, which coordinate system is registered with the coordinate system of the image acquisition device. In this context an angle in the horizontal plane and an angle relative to vertical (thus relative to the direction of the force of gravity) are also determined. At this point it is noted that instrument trajectories or guide paths can also be considered in which the instrument should take up two different directions. This means that the direction is changed during the procedure. Two angles must then be correspondingly, respectively considered.

The patient table and the orientation device are now mechanically positioned so that the entrance point is marked by the laser of a first indicator unit. The laser marking can thereby be a single point of an individual laser; however, a crosshair is also conceivable which can be generated by the intersection of two or more laser fans, for example. The second fan of such a first indicator unit can be selected to be notably narrower in its fan width when a second indicator unit with a second laser fan is to be used to indicate the direction later.

The correct positioning of the patient table in relation to the orientation device can be achieved by moving the patient table, typically along the head-to-foot direction of the patient; by moving the orientation device and/or the laser; and/or by a combination of the preceding possibilities. The relative position of the orientation device in relation to the patient table (and therefore to the acquired image data) is typically determined by a position sensor of the table itself, however also conceivably by means of physically present markings on the table or the orientation device that are read by the treating personnel.

The direction along the angle in the horizontal plane is then marked by rotating the first indicator unit (and thus the laser fan). This can take place via corresponding angular position sensors/distance measurements by means of the orientation device itself; however, physical markings provided on the patient table or the orientation device are also conceivable in turn, which markings can be read by the treating personnel and be used for a correct adjustment.

The angle relative to vertical is then correctly indicated in any case with the use of the inclination sensor. The guidance of the treating personnel can then take place either via a second indicator unit (which can likewise emit a laser fan), wherein the inclination sensor then measures the angle of the second indicator unit in relation to vertical, or can also take place by using an instrument guide, wherein the inclination sensor then determines the angle of the instrument guide in relation to vertical.

At this point it is further noted that, if multiple optical indicator unit are used, the color of the light (in particular of the laser) can naturally be selected differently in order to enable a differentiation between the light of the different indicator units.

Furthermore, it is noted that the adaptation of the table position and the alignment of the components of the orientation device or the instrument guide can in principle be implemented manually. However, actuators (in particular stepper motors or the like) are naturally also conceivable that can enable an automatic adjustment. In the case of manual operation, the treating personnel can be provided with feedback, either from the orientation device or the instrument guide itself or via a monitor. The feedback can take place numerically, via color, text-based and/or even acoustically.

Finally, it is noted that in the event that a laser is used in order to indicate the angle relative to vertical, the laser beam can also be used in order to determine the distance from the medical instrument via a laser distance measurement. Such a laser distance measurement can achieve an accuracy or precision of approximately 0.5 mm and ultimately provides a good indication of how deep the instrument has already been inserted into the patient.

In addition to the method, the present invention also concerns an orientation device to mark a predetermined guide path of a medical instrument (in particular a needle) having at least one adjustable indicator unit, in particular a light source. An orientation device according to the invention is characterized by having a one-dimensional or two-dimensional inclination sensor is to adjust the at least one indicator unit with regard to an angle of the guide path relative to vertical. The orientation device according to the invention is thus suitable to implement the method according to the invention; in particular, all statements made with regard to the method according to the invention apply analogously to the orientation device according to the invention. The orientation device according to the invention consequently also offers the advantage that incorrect positioning errors relative to vertical can largely be avoided due to the absolute ability to determine the vertical, thus the direction in which gravity acts. An improved marking of the guide path takes place in this manner, such that an improved orientation of the medical instrument follows.

As mentioned, the inclination sensor can be a spirit level and/or an accelerometer, in particular embodying a MEMS. An accelerometer is thereby preferred since highly accurate angles can hereby be obtained as measurement data.

The inclination sensor can be arranged at a needle guide that is used as an indicator unit or that includes the indicator unit. Since the position relative to vertical is fundamentally known due to the inclination sensor, an independently movable element of the orientation device can also be used in order to realize the indication of the angle relative to vertical.

Alternatively or additionally, the inclination sensor can be arranged at an optical indicator unit, in particular an indicator unit radiating a laser fan. It is advantageous for two indicator units to be provided to respectively radiate at least two independently adjustable laser fans, wherein the inclination sensor is associated with an indicator unit. An advantageous realization of such an orientation device having two independently adjustable indicator units with laser fans has a stand (mount) with a horizontal first carrier at whose ends are arranged (via a pivot coupling rotatable in a horizontal plane) a second carrier that can be displaced in translation and the first indicator unit. The second carrier can be tilted around a first horizontal axis counter to the first carrier. The second indicator unit is arranged at the second carrier, which can be tilted around a second axis parallel to the first horizontal axis and the inclination sensor. The two tilting possibilities around a horizontal axis ultimately serve to be able to compensate for displacement of the entrance point indicated on the skin of a patient, which displacement occurs given a tilting around a horizontal axis. Ultimately, the second tilting capability around a horizontal axis (which can be realized by a hinge coupling) can thus be regarded as a type of compensation means. In this context it is advantageous for the first indicator unit to already unambiguously define the entrance point, since then an important orientation is hereby provided for the treating personnel given a manual adjustment of the second indicator unit, such that both the correct entrance direction and the correct entrance point are indicated. If the correct adjustment is made, the overlap line of the two laser fans thus indicates the correct guide path; if the two laser fans are of light of different colors, for the treating personnel it is also immediately apparent in what direction a correction must be made since only one color is visible on the instrument or an instrument mount. The first indicator unit can be designed to mark a point, such that this indicator unit radiates two fans that are situated perpendicular to one another and spread out with different widths, which fans mark a point. Moreover, in this embodiment it is also appropriate for two indicator units to be used that each radiate a fan, with the indicated units being oriented relative to one another so that the laser fan of the first indicator unit and the laser fan of the second indicator unit are situated basically perpendicular to one another.

### BRIEF DESCRIPTION OF THE DRAWINGS

FIG. 1 schematically illustrates an orientation device according to the invention.

FIG. 2 shows a treatment arrangement that includes the orientation device according to the invention.

FIGS. 3 through 5 are schematic illustrations to explain the method according to the invention.

FIG. 6 schematically illustrates an orientation device of a second embodiment according to the invention.

FIG. 7 schematically illustrates the method according to the invention using the second embodiment of the orientation device.

### DESCRIPTION OF THE PREFERRED EMBODIMENTS

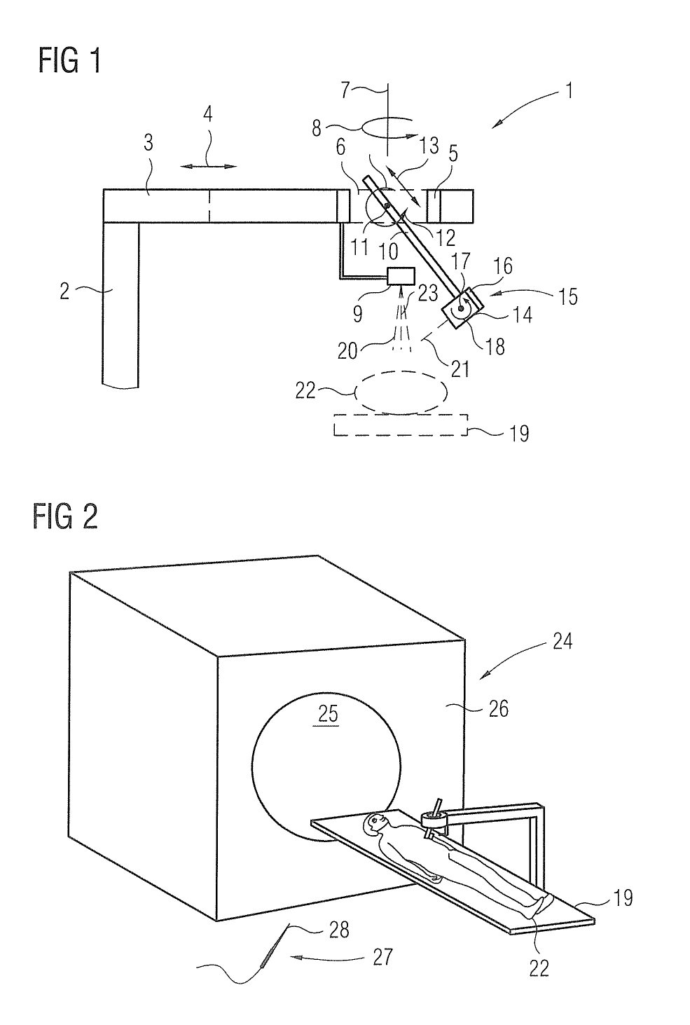

FIG. 1 is a schematic drawing of a vertical section through an orientation device 1 of a first embodiment according to the invention. A first carrier 3 is supported on a stand 2. It is variable in terms of its length (see arrow 4). In the first carrier 3 a rotating disc 5 with a central through hole [inner width] 6 is admitted that can be rotated around a vertical axis 7. The rotating disc 5 now supports a first indicator unit 9 as well as a second carrier 10 on the other hand.

The second carrier 10 is mounted so that it can be tilted both around a horizontal axis 11 (arrow 12) and can be longitudinally displaced in the mount (arrow 13).

A second indicator unit 14 with an inclination sensor 15 (which here is fashioned as an accelerometer 16 that is based on MEMS technology) is now also attached to the second carrier 10, for example via a joint. The second indicator unit 14 with the inclination sensor 15 can be pivoted around a second horizontal axis 17 that is parallel to the first horizontal axis 11 (arrow 18). Overall six degrees of freedom result together with the longitudinal movement of a patient table 19 (only indicated here) in order to adjust the orientation device so that an entrance point of a medical instrument and its entrance direction (thus summarized as the guide path) can be spatially marked for a user.

For this purpose, the indicator unit 9, 14 (each formed by a diode laser) are fashioned to radiate laser fans 20, 21 perpendicular to one another. For example, a cylindrical lens can be provided in a known manner to generate each laser fan.

In the present case the indicator unit 9, 14 use light of different colors, namely bright green and bright red. If the two fans 20, 21 intersect, they mark a straight line—the provided guide path. There the light of the two fans 20, 21 mix to form bright yellow. If a medical instrument (for example a needle or an instrument guide) is held in the fan region, it can consequently be seen (due to the reflection at the instrument surface or, respectively, guide surface) whether the instrument/the guide is oriented in the correct entrance direction.

The intersection point of the two fans on the skin of a patient 22 (only indicated here) on the patient table 19 marks the predetermined entrance point. As is easily visible from FIG. 1, in the adjustment of the vertical angle of the second carrier 10 this can be displaced so that—in addition to the fan 20—the first indicator unit 9 radiates a second, very much narrower fan 23 situated perpendicular to the fan 20, which is suitable to also already by itself mark the entrance point and allow an adjustment of the second carrier 10 so that the correctly predetermined entrance point is maintained.

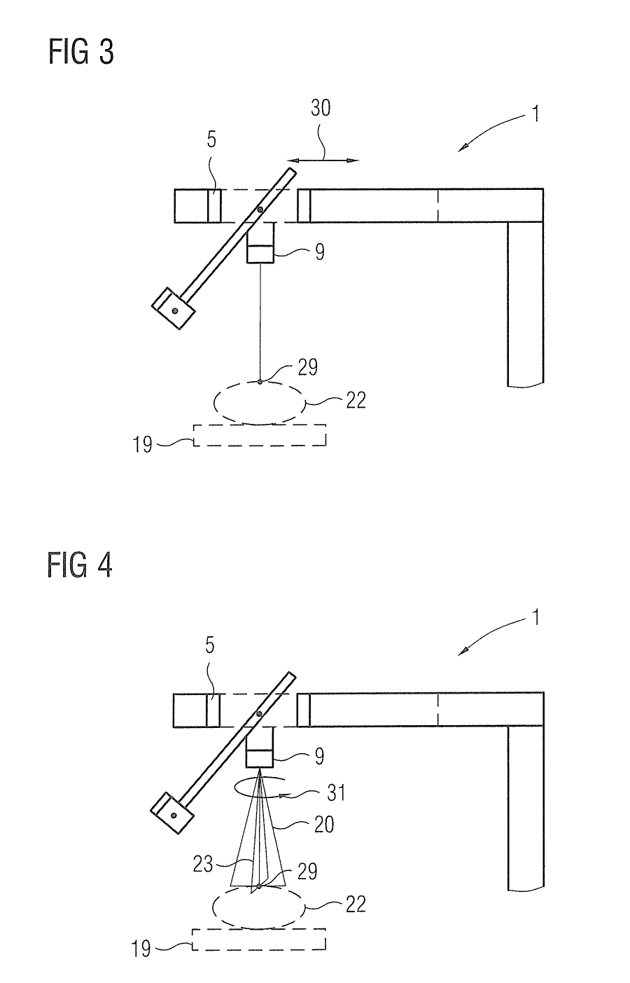

FIG. 2 now shows a treatment arrangement 24 that includes the orientation device 1 (only schematically shown). The orientation device 1 is arranged outside of a patient receptacle 25 of an image acquisition device 26, in particular of a CT device or an MR device. The patient table 19 with the patient 22 arranged on it can be moved out of the patient receptacle 25 into the region of the orientation device 1. It is thus possible for a treating personnel to correctly place a medical instrument 27 (here a needle 28) at an entrance point and to introduce it in the correct entrance direction, as should now be explained in detail using FIGS. 3-5.

However, image data of the patient 22 are initially acquired with the image acquisition device 26. The image data are used in order to plan the impending procedure, which means that the guide path of the instrument 27 (in particular the planned entrance point 29 and the entrance direction) are determined. These are now consequently known in the coordinate system of the image acquisition device 26 with which the orientation device 1 is registered or can be registered.

The adjustment steps described in the following can in principle be implemented manually by the treating personnel, for example directed via a monitor or via other auxiliary means. However, it is just as easily possible to provide an actuator means (not shown in detail) for each provided degree of freedom of movement (this is provided for the most part anyway for the patient table 19), which actuator means can be actuated by a central control device in order to enable an automatic alignment of the patient table and of the components of the orientation device 1. Architectures for automatic adjustment of components are well known and do not need to be described herein.

In a first step the patient table 19 is now initially moved so that the broad fan 20 of the first indicator unit 9 runs through the planned entrance point 29. The first carrier 3 is then adjusted in terms of its length so that the fans 20, 23 mark the predetermined entrance point 29 in the manner of a cross-hair. This is indicated by the arrow 30 in FIG. 3.

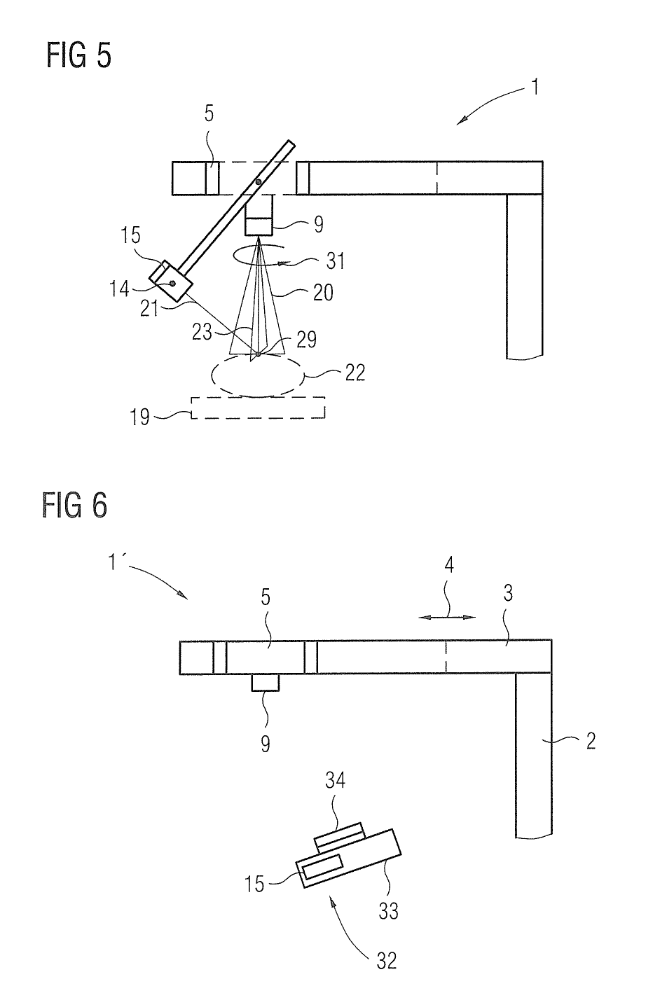

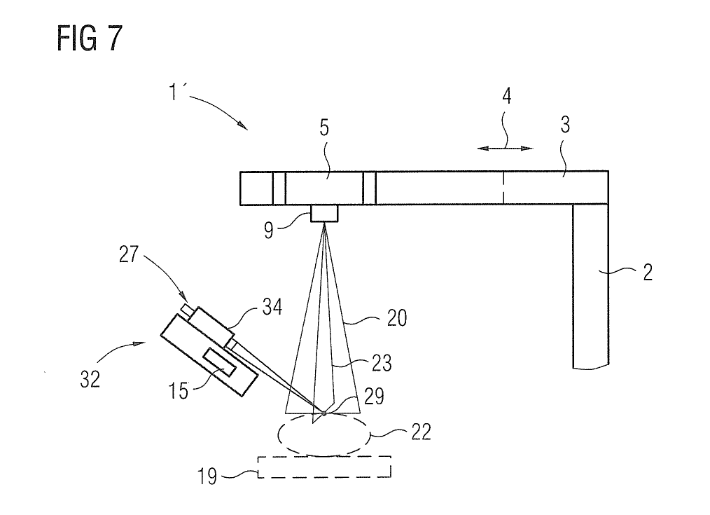

Finally, as explained by FIG. 5 the angle is adjusted relative to vertical, which is ultimately determined by the angle of the fan 21 of the second indicator unit 14. The registration data are not used for this; rather, only the measurement data of the inclination sensor 15 are used. This indicates how the direction of gravitation is situated relative to the indicator agent 14. By means of the two tilting possibilities of the second carrier 10 the indicator unit 14 is adjusted so that, the fan 21 furthermore marks the planned entrance point 29 but also so that the angle relative to vertical corresponds to the predetermined angle. The overlap line of the fans 20, 21 then marks the planned guide path of the instrument 27, wherein the intersection point of the fans 20, 21 on the patient 22 marks the entrance point 29. For the treating personnel it is now easy to correctly and precisely orient the instrument 27, in particular since (due to the inclination sensor 15) the angle relative to vertical can be set exactly and with high precision at the orientation device 1, independent of incorrect positions.

FIG. 6 shows a second embodiment of an orientation device 1′ according to the invention. For simplicity, corresponding components are provided with the corresponding reference characters. In this second embodiment the second carrier 10 with the second indicator unit 14 is clearly no longer required; the first indicator unit 9 can be arranged directly at the rotating disc 5 or another pivot coupling. Instead of this, an instrument guide 32 (here a needle guide 33) is now provided that has a receptacle 34 for the needle 28. The inclination sensor 15 is also now provided in or on the instrument guide 32.

This modifies the method according to the invention as follows. The initial steps up to the adjustment of the angle in the horizontal plane (FIG. 4) remain the same. The instrument 27 directed in the instrument guide 32 is then placed at the entrance point (marked by the laser fans 20, 32) and oriented in the horizontal direction that is marked by the fans 20. Using the measurement data of the inclination sensor 15 it is then checked whether the correct angle relative to vertical is present. If this is not the case, the treating personnel is provided (via an output means, in particular an optical and/or acoustic output means) with information as to in which direction to tilt, such that the treating personnel is directed (based on the angle measurement with the inclination sensor 15, which moreover is in turn realized as an accelerometer based on MEMS technology) step by step to the correct entrance direction.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for marking a predetermined guide path for a medical instrument to be introduced into a patient, said method comprising the steps of:
with an indicator unit, generating a representation of a guide path for a medical instrument to be introduced into a patient, said guide path being at an angle relative to vertical; and
with an inclination sensor selected from the group consisting of one-dimensional inclination sensors and two-dimensional inclination sensors, obtaining and emitting measurement data allowing adjustment of said indicator unit, and the indication of said guide path emitted thereby, with respect to said angle relative to vertical.

2. A method as claimed in claim 1 comprising employing a spirit level as said inclination sensor.

3. A method as claimed in claim 1 comprising employing an accelerometer as said inclination sensor.

4. A method as claimed in claim 3 comprising employing an accelerometer comprising a MEMS as said inclination sensor.

5. A method as claimed in claim 1 comprising adjusting said indicator unit solely using said measurement data from said inclination sensor.

6. A method as claimed in claim 1 comprising describing a previously determined direction of said guide path with an angle representing rotation in a horizontal plane and said angle relative to vertical.

7. A method as claimed in claim 1 comprising embodying said inclination sensor in an instrument guide that guides said instrument.

8. A method as claimed in claim 7 comprising employing a light source as said indicator unit and emitting a light beam as said representation of said guide path.

9. A method as claimed in claim 8 comprising emitting said light beam as a laser fan.

10. An orientation device for marking a predetermined guide path for a medical instrument to be introduced into a patient, comprising:
an indicator unit that generates a representation of a guide path for a medical instrument to be introduced into a patient, said guide path being at an angle relative to vertical; and
an inclination sensor selected from the group consisting of one-dimensional inclination sensors and two-dimensional inclination sensors, that obtains and emits measurement data allowing adjustment of said indicator unit, and the indication of said guide path emitted thereby, with respect to said angle relative to vertical.

11. An orientation device as claimed in claim 10 wherein said inclination sensor is a spirit level.

12. An orientation device as claimed in claim 10 wherein said inclination sensor is an accelerometer.

13. An orientation device as claimed in claim 12 wherein said accelerometer comprises a MEMS.

14. An orientation device as claimed in claim 10 wherein said indicator unit is adjustable using solely said measurement data from said inclination sensor.

15. An orientation device as claimed in claim 10 wherein a previously determined direction of said guide path is described with an angle representing rotation in a horizontal plane and said angle relative to vertical.

16. An orientation device as claimed in claim 10 comprising an instrument guide that guides said instrument, with said inclination sensor embodied in said instrument guide.

17. An orientation device as claimed in claim 16 wherein said indicator unit is a light source that emits a light beam as said representation of said guide path.

18. An orientation device as claimed in claim 17 wherein said light source emits said light beam as a laser fan.
